(19) United States
(12) Patent Application Publication
(10) Pub. No.: US 2024/0065303 A1
(43) Pub. Date: Feb. 29, 2024

(54) BITTER MASKING AGENT

(71) Applicant: SOLUGEN, INC., Houston, TX (US)

(72) Inventors: Jun Su An, Houston, TX (US); Jason Helander, Richmond, TX (US); Frederyk Ngantung, Houston, TX (US); LoongYi Tan, Houston, TX (US); Catherine Gonzalez, Houston, TX (US)

(73) Assignee: SOLUGEN, INC., Houston, TX (US)

(21) Appl. No.: 18/269,238

(22) PCT Filed: Mar. 29, 2021
(86) PCT No.: PCT/US2021/024743

§ 371 Date: Jun. 22, 2023

Related U.S. Application Data
(60) Provisional application No. 63/129,331, filed on Dec. 22, 2020.

(51) Int. Cl. A23L 27/00; A21D 2/14; A23F 5/24; A61K 31/00; A61K 47/12

(52) U.S. Cl. CPC A23L 27/84; A21D 2/145; A23F 5/24; A23L 27/86; A61K 31/658; A61K 47/12

(57) ABSTRACT

A bitter masking agent includes a biochelant and a salt. A composition for an additive to reduce bitterness includes a chelant sodium salt including an aldonic, uronic, or aldaric acid. The bitter masking agent or additive comprising the biochelant and the salt can be combined with an edible material to reduce the bitterness thereof.

### CROSS-REFERENCE TO RELATED APPLICATIONS

This application is a 35 U.S.C. § 371 national stage application of PCT/US2021/024743 filed Mar. 29, 2021, and entitled “Bitter Masking Agent,” which claims benefit of U.S. provisional patent application Ser. No. 63/129,331 filed Dec. 22, 2020, and entitled “Bitter Masking Agent,” each of which is hereby incorporated herein by reference in its entirety for all purposes.

### TECHNICAL FIELD

The present disclosure relates generally to bitter masking agents. More specifically, the present disclosure relates to compositions to decrease bitterness, increase sweetness, and have minimal salt flavor profile; and ingestible and/or edible materials prepared from same.

### BACKGROUND

Bitter taste perception is supposed to have evolved as a mechanism that averts possible toxins ingested. Bitter masking agents (BMAs) are compounds that reduce, disguise, or block the bitter taste of food products. Apart from food products, BMAs have been widely used in beverage formulations. Further, BMAs have also proven to be beneficial for people sensitive to medicines. Specifically, taste is a crucial factor that determines the palatability of oral dosage forms of therapeutic agents and consequently patient compliance. Accordingly, BMAs are used in the pharmaceutical industry to reduce the bitterness of medicines, especially for children.

BMAs are also used to reduce the bitterness of products containing hydrolyzed soy, whey proteins, and menthol; and oral care products. All these factors are responsible for increasing the global demand for bitter masking agents.

### SUMMARY

Aspects of bitter masking agents are disclosed herein. In one embodiment disclosed herein, a bitter masking agent comprises a biochelant and a salt.

Aspects of compositions of additives to reduce bitterness are disclosed herein. In one embodiment disclosed herein, a composition for an additive to reduce bitterness comprises a chelant sodium salt comprising an aldonic, uronic, or aldaric acid.

### BRIEF DESCRIPTION OF THE DRAWINGS

For a more complete understanding of the present disclosure and the advantages thereof, reference is now made to the following brief description, taken in connection with the accompanying drawings and detailed description, wherein like reference numerals represent like parts.

FIG. 1 is a graph of the results of a taste test as described in Example 1.

FIG. 2 is a graph of bitterness level as a function of masking agent dose as described in Example 3.

FIG. 3 is a plot of the percent bitterness as a function of masking agent dose as described in Example 3.

FIG. 4 is a graph of the results of the taste test for the samples from Example 4.

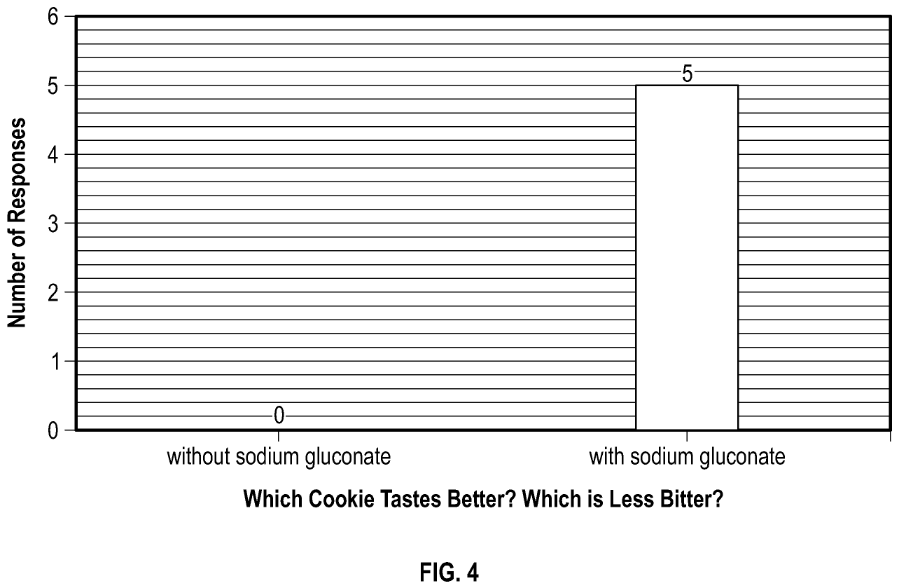

### DETAILED DESCRIPTION

There exists an ongoing need for effective BMAs able to improve the flavor profile of ingestible compositions. Disclosed herein are compositions for use as BMAs in an ingestible composition. Herein, “ingestible” has its ordinary and customary meaning as referring to a characteristic of being able to be taken into the body. In one or more aspects, the BMA is a component of an ingestible edible composition; alternatively, the BMA is a component of an ingestible non-edible composition.

In an aspect, the BMA comprises a biochelant and one or more metal salts. BMAs of the type disclosed herein may be included in an ingestible composition and improve the taste profile of the composition.

In an aspect, the BMA comprises a chelant. Herein, a chelant, also termed a sequestrant or a chelating agent, refers to a molecule capable of bonding a metal. The chelating agent is a ligand that contains two or more electron-donor groups so that more than one bond forms between a metal ion and the ligand. In an aspect, the chelant is a biochelant. As used herein, the prefix “bio” indicates that the chemical is produced by a biological process such as through the use of an enzyme catalyst.

In an aspect, the biochelant comprises aldonic acid, uronic acid, aldaric acid, or combinations thereof and a counter cation. In an aspect, the biochelant comprises a glucose oxidation product, a gluconic acid oxidation product, a gluconate, or combinations thereof. The glucose oxidation product, gluconic acid oxidation product, or combination thereof may be buffered to a suitable pH.

Alternatively, the biochelant comprises a buffered glucose oxidation product, a buffered gluconic acid oxidation product, or combinations thereof. In such aspects, the buffered glucose oxidation product, the buffered gluconic acid oxidation product, or combinations thereof are buffered to a suitable pH. In such aspects, the biochelant comprises a mixture of gluconic acid and glucaric acid, and further comprises a minor component species comprising n-keto-acids, C₂-C₆ diacids, or combinations thereof.

In an aspect, the biochelant comprises an aldonic acid, uronic acid, aldaric acid or combination thereof and a counter cation. The counter cation may comprise an alkali metal (Group 1), an alkali earth metal (Group II) or combinations thereof. In certain aspects, the counter cation is sodium, potassium, magnesium, calcium, strontium, cesium or a combination thereof.

In an aspect, the biochelant comprises Biochelate™ metal chelation product commercially available from Solugen, Inc. of Houston, Texas.

In one or more aspects, the biochelant is present in a finished food product in an amount of from about 0.05 weight percent (weight per volume w/v) to about 1 w/v based on the total weight of the finished food product, alternatively from 0.1% w/v about 0.5% w/v to about, or alternatively from about 0.25% w/v to about 1% w/v.

In an aspect, the BMA is included in an ingestible composition alone or as a component of a sweetener. Sweeteners refer to food additives that are used or intended to be used either to impart a sweet taste to food or as a tabletop sweetener. Nonlimiting examples of sweeteners include artificial sweeteners such as acesulfame potassium, alitame, aspartame, cyclamate, saccharin and sucralose; natural sweeteners such as monk fruit, stevioside, and thaumatin; caloric sweeteners such as maple syrup, honey and molasses; starch-derived compounds such as glucose and fructose; and sugar alcohols such as erythritol, mannitol, xylitol, sorbitol and tagatose. While these aforementioned sweeteners are effective, many of these artificial sweeteners are not without their downsides. Many of the intense sweeteners can have an undesired bitter taste if they are not formulated correctly. The addition of a BMA of the type disclosed herein can lead to a reduction in bitterness for these aforementioned sweeteners. In an aspect, the BMA is combined with a sweetener to form a sweetening composition comprising a BMA, designated herein as a SC-BMA.

In an aspect, the BMA and/or SC-BMA comprises a salt. Any salt suitable with the other components of the BMA and/or SC-BMA may be incorporated in an amount effective to meet some user and/or process goal. In an aspect, the salt comprises zinc sulfate, tripotassium phosphate, calcium chloride, magnesium chloride. potassium chloride, sodium chloride, sodium, potassium, magnesium, calcium, and zinc salts of carboxylic acids such as: gluconic, glucaric, citric, lactic, maleic, tartaric, or a combination thereof.

In an aspect, a BMA and/or SC-BMA of the type disclosed herein is included in an edible composition such as a food product. Herein “edible” refers to a material that is fit to be eaten as food. “Food” herein refers to any edible product intended for consumption by humans or animals. The term “food” and the term “food and beverage” are used herein interchangeably.

Examples of food and beverage products or formulations include, but are not limited to sweet coatings, frostings, or glazes for comestible products or any entity included in the Soup category, the Dehydrated and Culinary Food Category, the Dried Processed Food category, the Beverage category, the Ready Meal category, the Canned or Preserved Food category, the Frozen Processed Food category, the Chilled Processed Food category, the Snack Food category, the Baked Goods category, the Confectionary category, the Dairy Product category, the Ice Cream category, the Meal Replacement category, the Pasta and Noodle category, the Sauces, Dressings, Condiments category, the Baby Food category, the Spreads category or combinations thereof.

Exemplary foods include confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, powder mints, boiled sweets, pastilles, gums, jellies and chews, toffees, caramels and nougat, medicated confectionery, lollipops, liquorice, other sugar confectionery, gum, chewing gum, sugarized gum, sugar-free gum, functional gum, bubble gum, cough drops, herbs, seeds, spices, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, ready to eat cereals, family breakfast cereals, flakes, muesli, other cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yogurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, fruit juices, vegetable juices, cream, cheese, processed cheese, spreadable processed cheese, unspreadable processed cheese, unprocessed cheese, spreadable unprocessed cheese, hard cheese, packaged hard cheese, unpackaged hard cheese, yogurt, plain/natural yogurt, flavored yogurt, fruited yogurt, probiotic yogurt, drinking yogurt, regular drinking yogurt, probiotic drinking yogurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded cereals and snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, yeast-based spreads, toppings, and syrups.

In an aspect, a BMA and/or SC-BMA is included in a non-edible ingestible product. Herein, “non-edible products” refer to supplements, nutraceuticals, functional food products (e.g., any fresh or processed food claimed to have a health-promoting and/or disease-preventing properties beyond the basic nutritional function of supplying nutrients), pharmaceutical and over the counter products, oral care products such as dentifrices and mouthwashes, cosmetic products such as sweetened lip balms and other personal care products that use sucralose and/or other sweeteners. In general, over the counter (OTC) product and oral care product generally refer to a product for household and/or personal use which may be sold without a prescription and/or without a visit to a medical professional.

Examples of the OTC products include but are not limited to vitamins and dietary supplements; topical analgesics and/or anesthetic; cough, cold and allergy remedies; antihistamines and/or allergy remedies; and combinations thereof. Vitamins and dietary supplements include, but are not limited to vitamins, dietary supplements, tonics/bottled nutritive drinks, child-specific vitamins, dietary supplements, any other products of or relating to or providing nutrition, and combinations thereof. Topical analgesics and/or anesthetic include any topical creams/ointments/gels used to alleviate superficial or deep-seated aches and pains, e.g., muscle pain; teething gel; patches with analgesic ingredient; and combinations thereof. Cough, cold and allergy remedies include, but are not limited to decongestants, cough remedies, pharyngeal preparations, medicated confectionery, antihistamines and child-specific cough, cold and allergy remedies, and combination products. Antihistamines and/or allergy remedies include but are not limited to any systemic treatments for hay fever, nasal allergies, insect bites and stings. Examples of oral care product include, but are not limited to mouth cleaning strips, toothpaste, toothbrushes, mouthwashes/dental rinses, denture care, mouth fresheners, at-home teeth whiteners and dental floss.

In an aspect, a BMA and/or SC-BMA is included in a coffee-containing beverage such as a hot or cold-brewed coffee or coffee formulation. Current techniques to reduce the bitterness profile of a coffee formulation include the incorporation of flavor-enhancing additives or by re-brewing. However, on a large-scale manufacturing scale, costs associated with additives and re-brewing will be substantial. A coffee-containing beverage incorporating a BMA and/or SC-BMA may result in a beverage that exhibits a taste profile that is smoother, less bitter, and less sour.

In an aspect, a BMA and/or SC-BMA is included in a cold-brew coffee formulation. In such aspects, the cold brew manufacturer can set wider specifications relating to its manufacturing process, which includes, but is not limited to coffee quality, coffee bean grind size, coffee steep/brew time, coffee steep/brew temperature and coffee concentration. The cold-brew coffee formulation comprising a BMA or SC/BMA may exhibit improved room temperature stability because of the reduced heat during production.

In an aspect, a BMA and/or SC-BMA is included in a cannabidiol (CBD)—containing beverage. CBD, short for cannabidiol, is one of more than 100 cannabinoids (or compounds) present in a cannabis plant. In an aspect, the cannabinoid comprises tetrahydrocannabinol, tetrahydrocannabinolic acid, cannabidiol, cannabinol, cannabigerol, cannabichromene, cannabicyclol, cannabivarin, tetrahydrocannabivarin, tetrahydrocannabiphorol, cannabidivarin, cannabichromevarin, cannabigerovarin, cannabigerol monomethyl ether, cannabielsoin, cannabicitran, or combinations thereof.

CBD oil derives from the flowers, leaves, and stems of the plant, while hemp oil commonly comes from the seeds. Medically, CBD is verified as an effective treatment for seizures. Beyond that, other health benefits are speculative. For example, CBD has been credited with reducing stress and pain, reducing anxiety, and promoting sleep.

A limiting step when formulating CBD-based beverages is remedying the natural bitter taste associated with CBD oil. The bitter taste of CBD beverages can be amplified when the drinks are formulated with artificial sweeteners like stevia or erythritol. Addition of a BMA and/or SC-BMA in a CBD-containing beverage may result in a beverage formulation having an improved taste profile.

The BMA and/or SC-BMA may be introduced to an ingestible and/or edible composition in any amount compatible with the other components of the composition and sufficient to meet some user and/or process goal. In an aspect, the BMA and/or SC-BMA is introduced to an ingestible and/or edible composition in an effective amount. For example, a BMA and/or SC-BMA may be added to the finished food product at a ratio of from about 0.05 weight percent (wt. %) to about 1 wt. %.

In an aspect, the BMA and/or SC-BMA is introduced to an ingestible and/or edible composition in an amount sufficient to reduce the bitterness of the composition by equal to or greater than about 10%, alternatively equal to or greater than about 20%, alternatively equal to or greater than about 40% or alternatively equal to or greater than about 80%. In general, bitterness may be determined using any suitable methodology. For example, bitterness may be determined using ISO 5495 that describes a procedure for determining whether there exists a perceptible sensory difference or a similarity between samples of two products concerning the intensity of a sensory attribute. This test is also referred to as a directional difference test or a 2-AFC test (Alternative Forced Choice).

In an aspect, the BMA and/or SC-BMA is added to a coffee product or caffeine product. The resultant coffee product or caffeine product may display a reduced bitterness as determined following the study of Leksrisompong (2012) with an intensity of equal to or less than about 5 on a scale of 0 to 15 point.

### Additional Aspects

Aspects disclosed herein include:

A first aspect is a bitter masking agent comprising a biochelant and a salt.

A second aspect which is the agent of the first aspect wherein the biochelant comprises an aldonic acid, uronic acid, aldaric acid, or combinations thereof.

A third aspect which is the agent of any of the first through second aspects wherein the biochelant comprises a buffered glucose oxidation product, a buffered gluconic acid oxidation product or combinations thereof.

A fourth aspect which is the agent of the third aspect wherein the buffered glucose oxidation product, the buffered gluconic acid oxidation product or combinations thereof further comprises n-keto-acids, C₂-C₆ diacids, or combinations thereof.

A fifth aspect which is the agent of any of the first through fourth aspects wherein the salt comprises zinc sulfate, tripotassium phosphate, calcium chloride, magnesium chloride. potassium chloride, sodium chloride, sodium, potassium, magnesium, calcium, and zinc salts of carboxylic acids such as: gluconic, glucaric, citric, lactic, maleic, tartaric, or a combination thereof.

A sixth aspect which is a sweetener comprising a sweetening agent and the bitter masking agent of any of the first through fifth aspects.

A seventh aspect which is the sweetener of the sixth aspect wherein the sweetening agent comprises acesulfame potassium, alitame, aspartame, cyclamate, saccharin, sucralose, monk fruit, stevioside and thaumatin; maple syrup, honey, molasses, glucose, fructose, erythiol, mannitol, xylitol, sorbitol, tagatose, or combinations thereof.

An eighth aspect which is a composition comprising the bitter masking agent of the first aspect.

A ninth aspect which is a composition comprising the sweetener of the seventh aspect.

A tenth aspect which is an ingestible composition comprising the composition of the seventh aspect.

An eleventh aspect which is the ingestible composition of the tenth aspect that is edible.

A twelfth aspect which is the ingestible composition of the tenth aspect that is inedible.

A thirteenth aspect which is a caffeinated beverage comprising the composition of any of the first through seventh aspects.

A fourteenth aspect which is a cannabinoid-containing ingestible product comprising the composition of any of the first through seventh aspects.

A fifteenth aspect which is a cannabinoid-containing ingestible product comprising tetrahydrocannabinol, tetrahydrocannabinolic acid, cannabidiol, cannabinol, cannabigerol, cannabichromene, cannabicyclol, cannabivarin, tetrahydrocannabivarin, tetrahydrocannabiphorol, cannabidivarin, cannabichromevarin, cannabigerovarin, cannabigerol monomethyl ether, cannabielsoin, acannabicitran, or a combination thereof.

A sixteenth aspect which is a composition for an additive to reduce bitterness comprising a chelant sodium salt comprising an aldonic, uronic, or aldaric acid;

A seventeenth aspect which is the composition of the sixteenth aspect wherein the composition is added to a food wherein the food comprising coffee or caffeine.

An eighteenth aspect which is the composition of any of the sixteenth through seventeenth aspects wherein the food is a sweetener.

A nineteenth aspect which is the composition of the eighteenth aspect wherein the sweetener is aspartame, sucralose, saccharin, stevia, erythritol, sorbitol, xylitol, or mannitol.

A twentieth aspect which is the composition of any of the sixteenth through eighteenth aspects wherein the food is a mineral salt.

A twenty-first aspect which is the composition of any of the sixteenth through twentieth aspects, wherein the salt is zinc sulfate, tripotassium phosphate, calcium chloride, magnesium chloride, potassium chloride, sodium chloride, sodium, potassium, magnesium, calcium, and zinc salts of carboxylic acids such as: gluconic, glucaric, citric, lactic, maleic, tartaric or a combination thereof.

A twenty-second aspect which is the composition of any of the sixteenth through twenty-first aspects wherein the chelant is comprised of glucaric acid, gluconic acid, glucuronic acid, glucose oxidation products, and gluconic acid oxidation products.

A twenty-third aspect which is the composition of any of the sixteenth through twenty-second aspects, wherein the chelant is comprised of sugar oxidation products comprising of disaccharides, oxidized disaccharides, uronic acid, and aldaric acid.

A twenty-fourth aspect which is the composition of any of the sixteenth through twenty-third aspects, wherein the chelant is comprised of gluconic acid, glucaric acid, glucuronic acid, n-keto-acids and C₂-C₆ diacids.

A twenty-fifth aspect which is the composition of any of the sixteenth through twenty-fourth aspects, wherein the counter-cation comprises of an alkali earth metal of group 1 and group 2.

A twenty-sixth aspect which is the composition of any of the sixteenth through twenty-fifth aspects, wherein in the food is a cannabinoid.

A twenty-seventh aspect is the composition of any of the sixteenth through twenty-sixth aspects, wherein the cannabinoid is tetrahydrocannabinol, tetrahydrocannabinolic acid, cannabidiol, cannabinol, cannabigerol, cannabichromene, cannabicyclol, cannabivarin, tetrahydrocannabivarin, tetrahydrocannabiphorol, cannabidivarin, cannabichromevarin, cannabigerovarin, cannabigerol monomethyl ether, cannabielsoin, and cannabicitran.

A twenty-seventh aspect which is a composition for an additive to reduce bitterness in food. The composition comprising a chelant sodium salt comprising an aldonic, uronic, or aldaric acid.

A twenty-eight aspect which is the composition of the twenty-seventh aspect wherein the food is coffee, caffeine beverages, and other coffee beverages.

A twenty-ninth aspect which is the composition of the any of the twenty-seventh through twenty-eighth aspects wherein the food is a sweetener.

A thirtieth aspect which is the composition of the twenty-ninth aspect wherein the sweetener is aspartame, sucralose, saccharin, stevia, erythritol, sorbitol, xylitol, mannitol.

A thirty-first aspect which is the composition of any of the twenty-seventh through thirtieth aspects wherein the food is a mineral salt.

A thirty-second aspect which is the composition of the thirty-first aspect wherein the salt is zinc sulfate, tripotassium phosphate, calcium chloride, magnesium chloride. potassium chloride, sodium chloride, sodium, potassium, magnesium, calcium, and zinc salts of carboxylic acids such as: gluconic, glucaric, citric, lactic, maleic, tartaric or a combination thereof.

A thirty-third aspect which is the composition of any of the twenty-seventh through thirty-second aspects, wherein the chelant is a mixture of aldaric, uronic acids.

A thirty-fourth aspect which is the composition of any of the twenty-seventh through thirty-third aspects wherein the chelant is a mixture of aldaric, uronic acids, and their respective counter-cation.

A thirty-fifth aspect which is the composition of any of the twenty-seventh through thirty-fourth aspects wherein the chelant is comprised of glucaric acid, gluconic acid, glucuronic acid, glucose oxidation products, and gluconic acid oxidation products.

A thirty-sixth aspect which is the composition of any of the twenty-seventh through thirty-fifth aspects wherein the chelant is comprised of sugar oxidation products comprising of disaccharides, oxidized disaccharides, uronic acid, and aldaric acid.

A thirty-seventh aspect which is the composition of any of the twenty-seventh through thirty-sixth aspects wherein the chelant is comprised of gluconic acid, glucaric acid, glucuronic acid, n-keto-acids and C₂-C₆ diacids.

A thirty-eighth aspect which is the composition of any of the twenty-seventh through thirty-seventh aspects wherein the counter-cation comprises of an alkali earth metal of group 1 and group 2.

A thirty-ninth aspect which is the composition of the twenty-seventh aspect wherein in the food is a cannabinoid.

A fortieth aspect which is the composition of the thirty-ninth aspect wherein the cannabinoid is tetrahydrocannabinol, tetrahydrocannabinolic acid, cannabidiol, cannabinol, cannabigerol, cannabichromene, cannabicyclol, cannabivarin, tetrahydrocannabivarin, tetrahydrocannabiphorol, cannabidivarin, cannabichromevarin, cannabigerovarin, cannabigerol monomethyl ether, cannabielsoin, and cannabicitran.

### EXAMPLES

The subject matter having been generally described, the following examples are given as particular aspects of the disclosure and are included to demonstrate the practice and advantages thereof, as well as preferred aspects and features of the inventions. It should be appreciated by those of skill in the art that the techniques disclosed in the examples which follow represent techniques discovered by the inventors to function well in the practice of the inventions, and thus can be considered to constitute preferred modes for its practice. However, those of skill in the art should, in light of the present disclosure, appreciate that many changes can be made in the specific aspects which are disclosed and still obtain a like or similar result without departing from the scope of the inventions of the instant disclosure. It is understood that the examples are given by way of illustration and are not intended to limit the specification of the claims to follow in any manner.

### Example 1

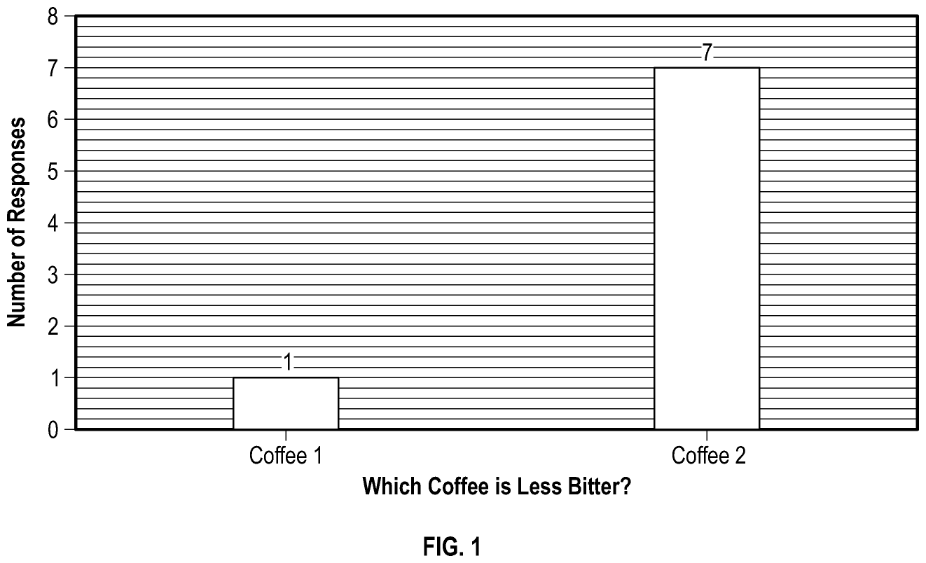

The ability of a BMA to reduce the bitterness of a coffee product was investigated. Specifically, varying amounts of a BMA according to aspects described herein was added to coffee samples. In a blind study, a panel of 8 individuals were asked to determine which coffee tasted less bitter. A graph of the results is presented in FIG. 1. As seen in FIG. 1, Coffee 2 was chosen as the sample that exhibited a less bitter, smoother taste. Coffee 2 had 0.5 grams of sodium gluconate added to a 200 g coffee.

### Example 2

In a blind study, a panel of 8 individuals were asked to determine which coffee tasted less bitter. FIG. 1 is a graph that shows the results from the panel. Referring to FIG. 1, coffee 2 was chosen as the sample which exhibited a less bitter, smoother taste. Coffee 2 had 0.5 grams of sodium gluconate added to a 200 g coffee.

### Example 3

The effect of sodium gluconate on bitterness as a dose response was investigated, In a blind study, six (6) trained panelists conducted a taste test with caffeine samples. As seen above, the caffeine bitter taste intensity references for a 5 and a 10, on the 0 to 15 point Spectrum intensity scale, were selected (Meilgaard et al., 2000). Caffeine solutions with and without sodium gluconate were prepared on a w/v basis with deionized water. Products were dispensed into lidded soufflé cups with 3-digit codes and evaluated at 21° C. Products were evaluated in duplicate by 6 trained panelists for bitter taste amelioration following the study by Leksrisompong et al. (2012).

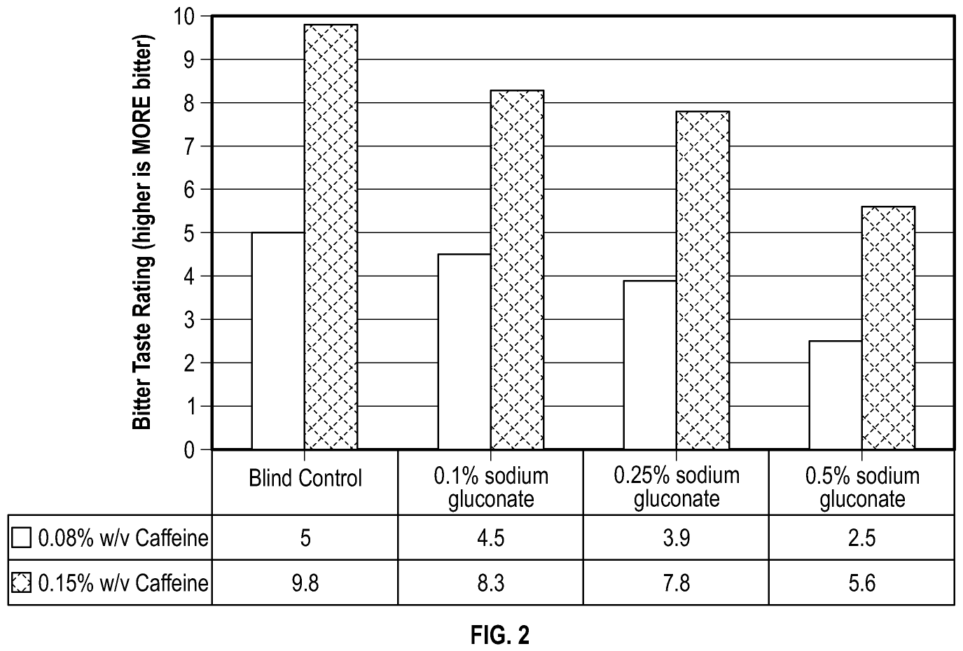

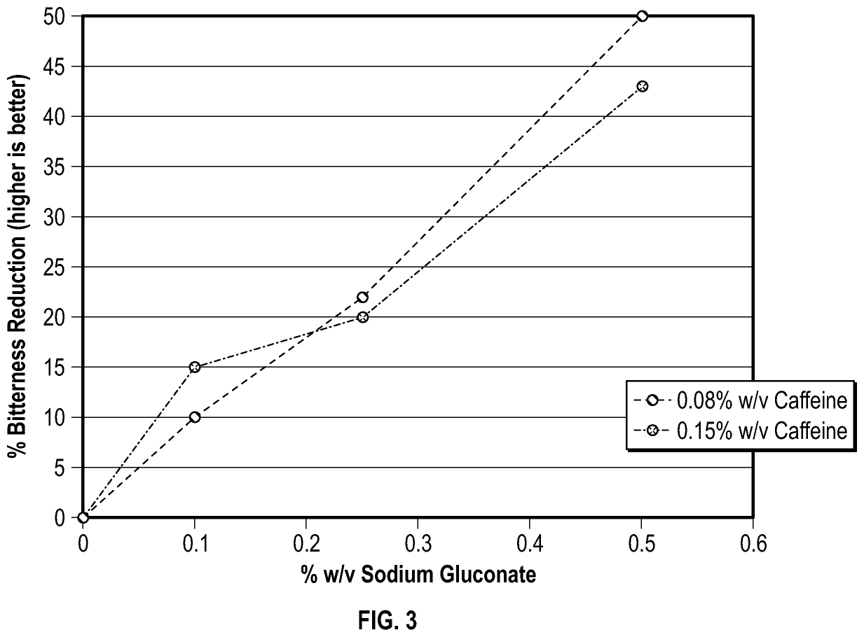

Referring to FIG. 2, the samples with sodium gluconate had significant bitterness reduction. Interestingly, the bitterness reduction seems to be linear in nature as seen in FIG. 3.

### Example 4

The level of sodium gluconate on bitterness reduction in baked goods was investigated. Using a commercially available recipe of chocolate chip cookies, a batch of cookies were baked and blind sampled. The batches were baked using the recipe (i) 2 sticks unsalted butter, at room temperature; (ii) 1 cup erythritol or 1 cup monk fruit sugar substitute or ½ cup stevia sugar substitute; (iii) for samples with sodium gluconate, ½ tsp of sodium gluconate was used for every cup of erythritol; (iv) 1¼ egg; (v) 1⅓ cup almond flour; (vi) 2 tsp vanilla extract; (vii) 1¼ tsp sea salt; (viii) ½ tsp baking powder; (ix) ¼ tsp baking soda; and (x) 1 bag Lily's Sweets Semi-Sweet Chocolate Chips or Milk Chocolate Baking Chips. The results from the tasting are displayed in FIG. 5. Five (5) people were brought in to compare batches with and without sodium gluconate. All 5 participants preferred the batch that contained sodium gluconate. All 5 participants reported that the batch without sodium gluconate had a bitter aftertaste.

While aspects of the disclosure have been shown and described, modifications thereof can be made without departing from the spirit and teachings of the presently disclosed subject matter. The aspects and examples described herein are exemplary only, and are not intended to be limiting. Many variations and modifications of the subject matter disclosed herein are possible and are within the scope of the present disclosure.

At least one aspect is disclosed and variations, combinations, and/or modifications of the aspect(s) and/or features of the aspect(s) made by a person having ordinary skill in the art are within the scope of the disclosure. Alternative aspects that result from combining, integrating, and/or omitting features of the aspect(s) are also within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, 5, 6, . . . ; greater than 0.10 includes 0.11, 0.12, 0.13, 0.14, 0.15, . . . ). For example, whenever a numerical range with a lower limit, RI, and an upper limit, Ru, is disclosed, any number falling within the range is specifically disclosed. In particular, the following numbers within the range are specifically disclosed: R=RI+k* (Ru-RI), wherein k is a variable ranging from 1 percent to 100 percent with a 1 percent increment, i.e., k is 1 percent, 2 percent, 3 percent, 4 percent, 5 percent, . . . 50 percent, 51 percent, 52 percent . . . 95 percent, 96 percent, 97 percent, 98 percent, 99 percent, or 100 percent. Moreover, any numerical range defined by two R numbers as defined in the above is also specifically disclosed. Use of the term “optionally” with respect to any element of a claim means that the element is required, or alternatively, the element is not required, both alternatives being within the scope of the claim. Use of broader terms such as comprises, includes, and having should be understood to provide support for narrower terms such as consisting of, consisting essentially of, and comprised substantially of.

Accordingly, the scope of protection is not limited by the description set out above but is only limited by the claims which follow, that scope including all equivalents of the subject matter of the claims. Each claim is incorporated into the specification as an aspect of the present disclosure. Thus, the claims are a further description and are an addition to the detailed description of the presently disclosed subject matter.

1. A bitter masking agent comprising:
a biochelant; and
a salt.

2. The agent of claim 1, wherein the biochelant comprises an aldonic acid, uronic acid, aldaric acid, or a combination thereof.

3. The agent of claim 1, wherein the biochelant comprises a buffered glucose oxidation product, a buffered gluconic acid oxidation product, or a combination thereof.

4. The agent of claim 3, wherein the buffered glucose oxidation product, the buffered gluconic acid oxidation product or combinations thereof further comprises n-keto-acids, C₂-C₆ diacids, or a combination thereof.

5. The agent of claim 1, wherein the salt comprises zinc sulfate, tripotassium phosphate, calcium chloride, magnesium chloride. potassium chloride, sodium chloride, sodium, potassium, magnesium, calcium, and zinc salts of carboxylic acids such as: gluconic, glucaric, citric, lactic, maleic, tartaric or a combination thereof.

6. A sweetener comprising a sweetening agent and the bitter masking agent of claim 1.

7. The sweetener of claim 6, wherein the sweetening agent comprises acesulfame potassium, alitame, aspartame, cyclamate, saccharin, sucralose, monk fruit, stevioside and thaumatin; maple syrup, honey, molasses, glucose, fructose, erythiol, mannitol, xylitol, sorbitol, tagatose, or combinations thereof.

8. A composition comprising the bitter masking agent of claim 1.

9. A composition comprising the sweetener of claim 7.

10. An ingestible composition comprising the composition of claim 7.

11. The ingestible composition of claim 10 that is edible.

12. The ingestible composition of claim 10 that is inedible.

13. A caffeinated beverage comprising the composition of any of claims 1 to 7.

14. A cannabinoid-containing ingestible product comprising the composition of any of claims 1 to 7.

15. The cannabinoid-containing ingestible product of claim 14, further comprising tetrahydrocannabinol, tetrahydrocannabinolic acid, cannabidiol, cannabinol, cannabigerol, cannabichromene, cannabicyclol, cannabivarin, tetrahydrocannabivarin, tetrahydrocannabiphorol, cannabidivarin, cannabichromevarin, cannabigerovarin, cannabigerol monomethyl ether, cannabielsoin, acannabicitran, or a combination thereof.

16. A composition for an additive to reduce bitterness comprising:
a chelant sodium salt comprising an aldonic, uronic, or aldaric acid.

17. The composition of claim 16, wherein the composition is added to a food and wherein the food comprises coffee or caffeine.

18. The composition of claim 17, wherein the food is a sweetener.

19. The composition of claim 18, wherein the sweetener is aspartame, sucralose, saccharin, stevia, erythritol, sorbitol, xylitol, or mannitol.

20. The composition of claim 17, wherein the food is a mineral salt.

21. The composition of any of claims 16-20, wherein the salt is zinc sulfate, tripotassium phosphate, calcium chloride, magnesium chloride. potassium chloride, sodium chloride, a sodium, potassium, magnesium, calcium, and zinc salts of carboxylic acids; sodium gluconic, sodium glucaric, sodium citric, sodium lactic, sodium maleic, sodium tartaric, potassium gluconic, potassium glucaric, potassium citric, potassium lactic, potassium maleic, potassium tartaric, potassium, zinc lactate, or a combination thereof.

22. The composition of claim 16, wherein the chelant comprises glucaric acid, gluconic acid, glucuronic acid, glucose oxidation products, or gluconic acid oxidation products.

23. The composition of claim 16, wherein the chelant comprises a sugar oxidation product comprising of disaccharides, oxidized disaccharides, uronic acid, or aldaric acid.

24. The composition of claim 16, wherein the chelant comprises gluconic acid, glucaric acid, glucuronic acid, n-keto-acids and C₂-C₆ diacids.

25. The composition of claim 16, wherein the counter-cation comprises of an alkali earth metal of group 1 and group 2.

26. The composition of claim 16, wherein in the food is a cannabinoid.

27. The composition of claim 16, wherein the cannabinoid is tetrahydrocannabinol, tetrahydrocannabinolic acid, cannabidiol, cannabinol, cannabigerol, cannabichromene, cannabicyclol, cannabivarin, tetrahydrocannabivarin, tetrahydrocannabiphorol, cannabidivarin, cannabichromevarin, cannabigerovarin, cannabigerol monomethyl ether, cannabielsoin, cannabicitran or a combination thereof.

28. A method of preparing a composition, the method comprising:
combining a bitter masking agent comprising a biochelant and a salt with an edible material; and
forming an edible composition based on the combining.

29. The method of claim 28, further comprising:
reducing a bitterness of the edible material based on combining the bitter masking agent with the edible composition.
